(19) United States
(12) Patent Application Publication
(10) Pub. No.: US 2022/0018164 A1
(43) Pub. Date: Jan. 20, 2022

(54) Law Enforcement Restraint Device

(71) Applicants: Gregory Frank Walwer, Guilford, CT (US); James Alexander Walwer, Guilford, CT (US)

(72) Inventors: Gregory Frank Walwer, Guilford, CT (US); James Alexander Walwer, Guilford, CT (US)

(21) Appl. No.: 17/377,794

(22) Filed: Jul. 16, 2021

Related U.S. Application Data
(60) Provisional application No. 63/103,081, filed on Jul. 20, 2020.

(51) Int. Cl. E05B 75/00; G01S 19/01; A61M 5/20

(52) U.S. Cl. CPC E05B 75/005; A61M 2209/088; A61M 5/20; G01S 19/01

(57) ABSTRACT

A law enforcement restraint device consisting of an implement handle and hollow shaft containing two or more collapsed, deployable, and detachable locking cuffs. The shaft is long enough for a law enforcement officer to safely apply the restraint device to a violent, mentally ill, or non-compliant detainee. A handle trigger allows for the exposure of an uncollapsed open locking cuff at the end of the shaft, ready for deployment to the detainee. Another trigger within the cuff automatically causes the locking of the detachable cuff about the limb of the detainee once the trigger is depressed by contact with the detainee limb. A flexible cable attached to the first locked cuff causes the exposure of a second locking cuff to the deployable position at the end of the shaft when the shaft is pulled away from the detainee. Following the deployment of two cuffs to two limbs of the detainee, pulling the shaft away from the detainee shortens the distance between the cuffs and further restrains the detainee. Pulling the shaft away from the detainee further releases the end of the cable from the handle and shaft. A key is used to release the clamp that tightens the cable and to release the cuffs from the locked position. Following the release of the detainee, the locking cuffs can be collapsed and reloaded along with the connecting cable into the shaft of the device for repeated use.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND OF THE INVENTION

### Field of Invention

This invention relates to law enforcement restraint devices, specifically to such restraint devices which are applied easily and safely.

### Background of the Invention

Law enforcement restraint devices are evolving with advances in technology and social awareness of the potential for abusive law enforcement practices. Existing restraint devices include striking batons, hand cuffs, disposable cuffs and zip ties, taser and stun guns, head and neck restraints, pepper spray, police dogs, and most recently, bola wraps, all of which present difficulties and dangers in applying to resistant detainees. These devices supplement existing bodily restraint practices that are also inherently dangerous, such as choke, basket, supine, and prone holds to subdue violent, mentally ill, and non-compliant detainees.

As revealed in U.S. Pat. No. 6,615,622 B2 to MacAleese et al. (2003), others have invented a law enforcement restraint device where there is a handle and shaft in the form of a baton, and where internal cuffs are extracted to a deployable position at the ends of the baton, but the cuffs are flexible plastic loops that require the manual manipulation of the detainee extremities of hands or feet by the user into the loops prior to retraction and tightening of the loops. The device requires intimate and dangerous engagement of the detainee by the user, as evident in the description of preferred embodiment, which include descriptions of use such as, “The officer swings the invention at the suspect's right knee to knock him off balance,” and “ . . . the officer uses leverage to spin the suspect around,” and “ . . . the officer continues to turn the suspect away from him and uses the invention to help twist the suspect's left arm behind him,” and “ . . . the officer now places the invention in the small of the suspect's back to further immobilize him,” with several more steps involved in forcing the extremities of the suspect through the loops prior to retraction and tightening of the loops. Hand cuffs with open hinged rigid elements and locking components have been in use for more than 100 years, and avoid the problem of awkwardly forcing extremities through enlarged flexible loops of retractable restraint devices, but these restraint devices along with disposable cuffs and zip ties also face the problem of application in close proximity to potentially violent and resistant detainees. Taser and stun guns, as anticipated by Coakley et al. 1997, avoid the problem of close encounters with the detainee, but the application of these devices can result in serious injury to the detainee, as is the case with the use of striking batons, pepper spray, police dogs, head and neck restraints, and any of the available direct bodily restraint methods requiring physical engagement by the law enforcement officer. The bola wrap, or other projectile netting devices, as in U.S. Pat. No. 5,706,795 to Gerwig (1998), anticipates a launching device that projects a net or bola to apprehend a detainee, but the invention faces the difficulty of accurately deploying the device against an individual at a distance, it may not reliably bind the limbs of the detainee to prevent fleeing, and further has the disadvantage of being imprecisely applied in a situation where the detainee may be in close proximity to innocent individuals or bystanders.

### Background of the Invention—Objects and Advantages

Accordingly, several objects and advantages of the present invention are to provide a law enforcement restraint device which consists of a hand-held implement containing a handle and shaft long enough to safely deploy collapsed detachable locking cuffs to a violent, mentally ill, or non-compliant detainee, with easily deployable and detachable locking cuffs bound by a flexible detachable cable with locking clamp allowing for tightening, thereby eliminating the need for dangerous bodily restraining devices or take-down techniques. Still further objects and advantages will become apparent from a consideration of the ensuing description and drawings.

### SUMMARY

In accordance with the present invention, a law enforcement restraint device comprises a handle and hollow shaft containing easily deployable and detachable locking cuffs bound by a detachable flexible cable with locking clamp for tightening.

### DRAWINGS—FIGURES

FIG. 1 shows the present invention and its relationship to the user and the detainee.

FIG. 2 shows the user applying the first locking cuff to the detainee.

FIG. 3 shows the deployment of the second cuff applied to the detainee.

FIG. 4 shows the tightening of the flexible cable connecting the cuffs.

FIG. 5 shows a telescoped cross-section view of the loaded restraint device.

FIG. 6 shows a telescoped cross-section view of the second uncollapsed locking cuff in the deployable position.

### DRAWINGS—REFERENCE NUMERALS

| | |
|---|---|
| 10 | law enforcement officer |
| 12 | detainee |
| 14 | restraint device |
| 16 | belt |
| 18 | handle |
| 20 | hollow shaft |
| 22 | handle trigger |
| 24 | uncollapsed and detachable locking cuff |
| 26 | detainee limb |
| 28 | flexible cable |
| 30 | second detachable locking cuff |
| 32 | second detainee limb |
| 34 | hinge pin |
| 36 | trigger spring |
| 38 | spring catch |
| 40 | main shaft spring |
| 42 | trigger channel |
| 44 | spring catch disc plate |
| 46 | rear spring plate |
| 48 | first catch position |
| 50 | second catch position |
| 52 | plunger |
| 54 | plunger knob |
| 56 | plunger rear plate |
| 58 | plunger front plate |
| 60 | front cable knob |
| 62 | cable stop |
| 64 | cuff channels |
| 66 | cuff trigger |
| 68 | cuff base |
| 70 | cuff trigger spring |
| 72 | middle cogs |
| 74 | cuff catches |
| 76 | catch springs |
| 78 | trigger lock |
| 80 | locking clamp |
| 82 | key |

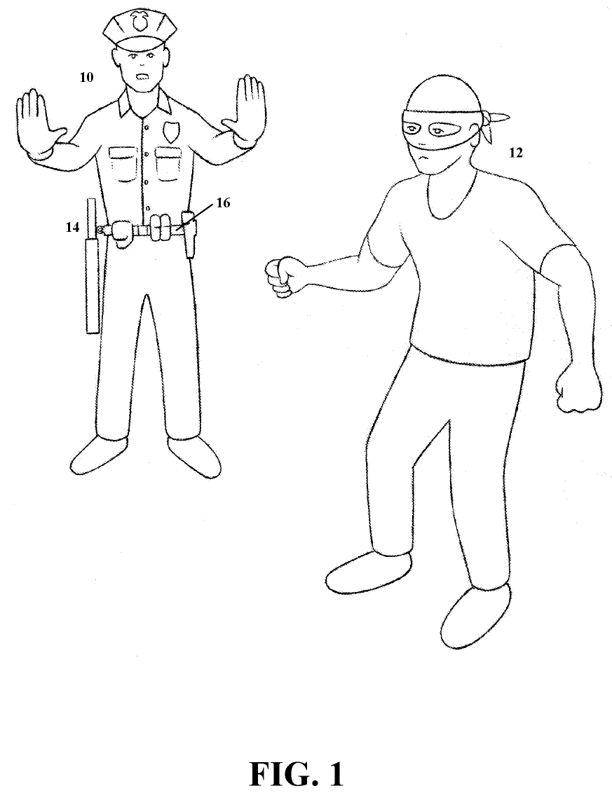

### Detailed Description—FIGS. 1, 2, 3, 4, 5, 6—Preferred Embodiment

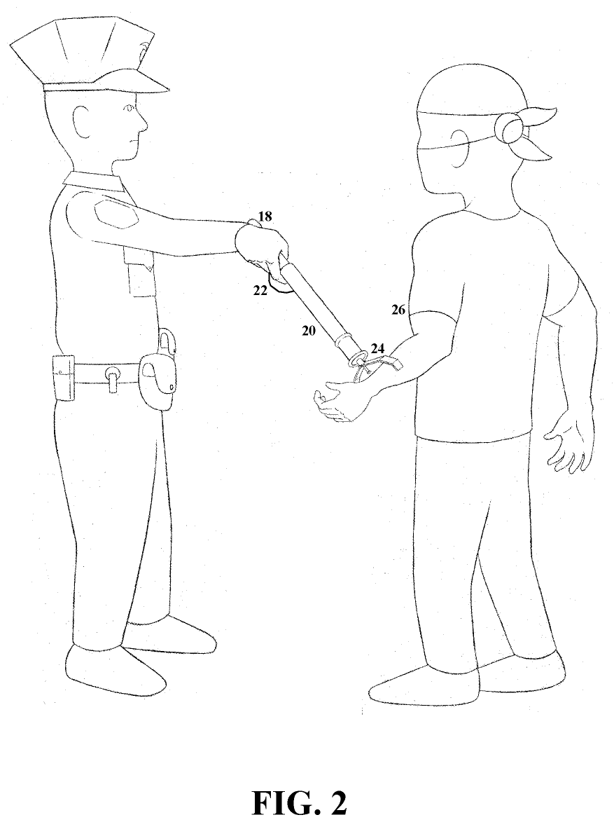

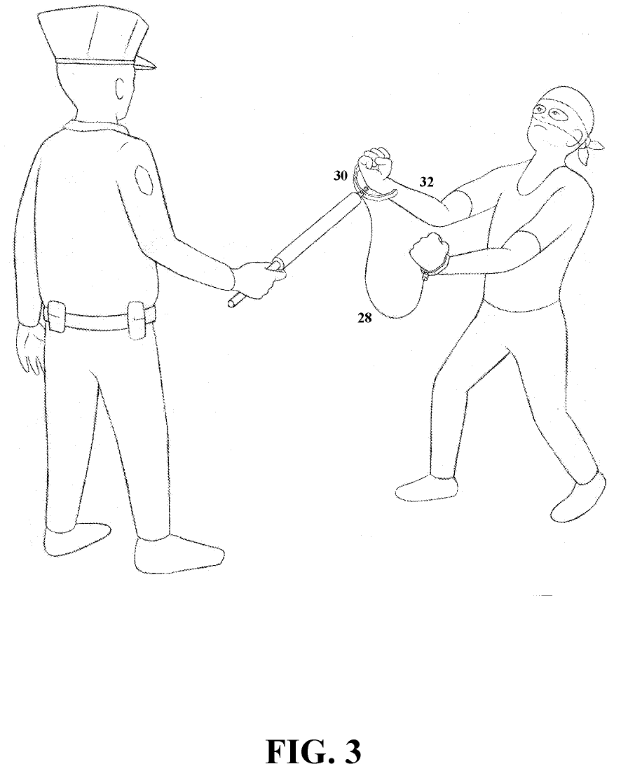

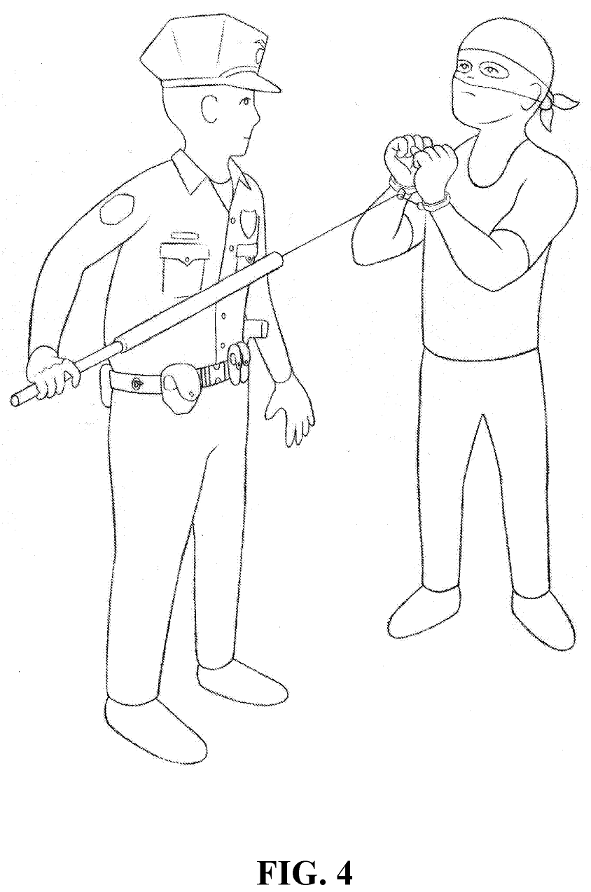

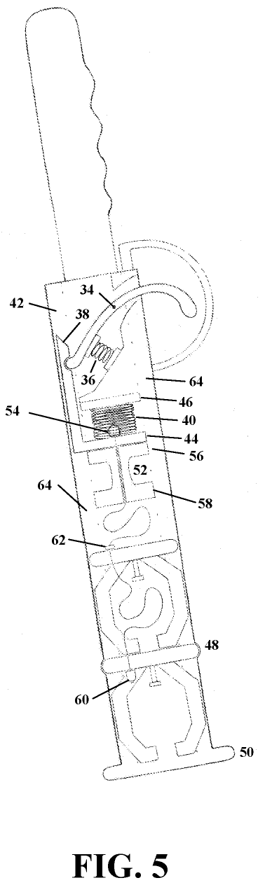

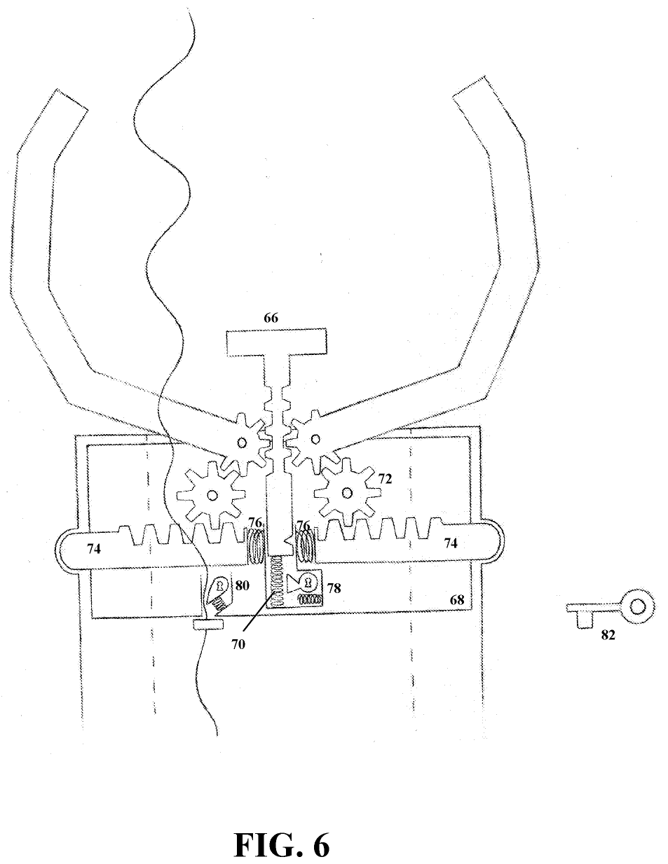

A preferred embodiment of the law enforcement restraint device is depicted in FIG. 1, FIG. 2, FIG. 3, and FIG. 4 (in use), and FIG. 5 and FIG. 6 (telescoped views showing internal components).

With reference to FIG. 1, the law enforcement officer 10 informs a detainee 12 that the restraint device 14 will be deployed for non-compliance with instructions for being arrested. As with a baton or hand cuffs, the restraint device is meant to be a portable item that can be worn on a belt 16 or otherwise detachably clipped to a uniform. In the preferred embodiment, with reference to FIG. 2, the restraint device consists of a handle 18 with hollow shaft 20 that contains collapsed locking cuffs and is long enough to safely apply the cuffs to the uncooperative detainee. A handle trigger 22 exposes at the end of the hollow shaft an uncollapsed and detachable locking cuff 24 in a deployable open position that can then be applied to a limb 26 of the detainee. With reference to FIG. 3, the applied and detached first cuff is connected by a flexible cable 28 to a second detachable locking cuff 30 that is drawn from a collapsed position within the hollow shaft of the handle to the opened deployable position at the end of the shaft by pulling the handle and shaft away from the detainee, who is already bound by the first detachable locking cuff. The second detachable locking cuff can then be applied to a second detainee limb 32 in the same manner as the first detachable locking cuff. With reference to FIG. 4, pulling the handle away from the detainee after applying the second cuff causes the flexible cable between the two cuffs to be tightened, thereby further binding the detainee.

With reference to FIG. 5 and a detailed cross-section view of the loaded restraint device, a slot in the handle allows for the trigger to rotate about a hinge pin 34 and is kept in place at rest via a trigger spring 36. When pulled, the trigger releases the barb of a spring catch 38, allowing the main shaft spring 40 to be released from a cocked position. A trigger channel 42 in the handle and hollow shaft ensures that the barbed spring catch does not obstruct other internal components of the cylinder of the hollow shaft. The main shaft spring is attached to the spring catch disc plate 44, and a separate rear spring plate 46 also serves as one wall of the chamber housing the trigger. The length of the main shaft spring is set so that its release results in the forward movement of the entire assembly of locking cuffs through the hollow shaft until the first detachable locking cuff is forced from the first catch position 48 to the second catch position 50. The flexible cable that joins the two locking cuffs has a plunger 52 that connects to the spring catch disc plate via a plunger knob 54 which can be forcibly pushed through the deformable spring catch disc plate, with a plunger rear plate 56 preventing it from being forced past the spring catch disc plate. The plunger front plate 58 is flush to the base of the second collapsed deployable and detachable locking cuff in the assembly when loaded, and in turn the front of the second collapsed deployable and detachable locking cuff is flush to the base of the first collapsed deployable and detachable locking cuff in the assembly when loaded. The front end of the flexible cable is secured to the base of the first collapsed deployable and detachable locking cuff via a front cable knob 60 that prevents its release. A cable stop 62 sets the maximum distance that the two cuffs can be apart, and further ensures that by pulling on the shaft after the first cuff is deployed, that the second cuff will be forcibly moved forward to the front deployable position at the end of the hollow shaft. Two cuff channels 64 in the hollow shaft of the device ensure that the collapsed deployable and detachable locking cuffs are set to the uncollapsed and deployable position at the front of the shaft in a predictable orientation to facilitate use, preferably perpendicular to the trigger shaft (although the trigger channel is shown as synonymous with one of the cuff channels in FIG. 5 for simplicity), and further to accommodate the size of the cuffs without increasing the overall diameter of the shaft to an unmanageable size. The cuff channels also further ensure that the arms of the cuffs are spaced apart correctly and are not accidentally locked closed when loading or reloading the components into the shaft.

With reference to FIG. 6 and a detailed cross-section view of the locking cuff, a cuff trigger 66 resides within the opened deployable cuff to cause the cuff to be forcibly locked and closed about the limb of the detainee when depressed, and simultaneously activates the release of the cuff from the end of the hollow shaft, via a system of cogs and toothed elements. In the preferred embodiment, each arm of the cuff has a hinged end mounted to a cuff base 68 that houses the internal components of the cuff. A cuff trigger spring 70 causes the forward positioning of the trigger and the simultaneous opening or uncollapsing of the cuff arms when exposed past the end of the hollow shaft. Middle cogs 72 articulate with the cogs of the cuff arms and toothed sections of cuff catches 74 that hold the cuff in the deployable position at the second catch position at the end of the hollow shaft. The second catch position is much larger than the first, so that only a slight expansion of the cuff catches and cuff arms is necessary to secure the first cuff when loading or reloading. Two catch springs 76 on either side of a cylinder containing the cuff trigger spring facilitate the positioning of the cuffs to the deployable position when forced forward by either the handle trigger in the case of the first cuff, or pulling the shaft away from the detainee in the case of the second cuff. The cogs on the cuff trigger articulate with cogs on the end of the cuff arms, so that when the trigger is depressed by forcibly being pushed against the limb of the detainee, the deployable cuff is forced fully closed and locked via a trigger lock 78. When the cable is pulled after the application of the second cuff to the detainee, the distance between the cuffs is shortened and a locking clamp 80 in the second cuff prevents loosening. A key 82 is used to loosen the locking clamp and release the cuffs locked by the trigger lock when appropriate. The unlocked, collapsed cuffs and connecting cord can be reinserted and loaded into the end of the shaft for future redeployment.

The preferred embodiment of FIGS. 1, 2, 3, 4, 5, and 6 shows aspects of the invention related to ease of use and safe application. Other embodiments could also be derived to accomplish these same goals. In alternative embodiments, more than two cuffs could be available to decrease the potential mobility of the detainee to resist or flee, and could also be used to bind more than one detainee together. Further, instead of a cuff trigger to apply and release the locking cuff, a handle trigger could be used to shoot a cuff towards the detainee as a projectile where a greater need for safety distance is necessary. In the preferred embodiment, the detachable locking cuffs are constructed of a rigid material such as stainless steel, while in the alternative embodiments, a more flexible material effectively serving as a slap bracelet could be used. In the preferred embodiment, an ideal length of the handle shaft is set to provide safety to the law enforcement officer, while alternative embodiments can include retractable or extendable shafts selected for particular arrest situations. In another alternative embodiment, the locking of the cuffs activates an internal GPS device that allows for law enforcement to track the detainee in the event of flight during or after the application of the cuffs. In yet another alternative embodiment, the trigger contains a sedative to further immobilize the uncooperative detainee.

We claim:

1. A law enforcement restraint device, comprising:
a) a handle and hollow shaft;
b) collapsed detachable locking cuffs within the hollow shaft;
c) handle trigger that causes the first detachable locking cuff to become uncollapsed in a deployable position at the end of the hollow shaft;
d) cuff trigger that causes the first cuff to lock onto the limb of a detainee and detach it from the end of the hollow shaft;
e) flexible cable connecting the first deployed cuff to a second cuff with means to set the second cuff to a deployable position at the end of the hollow shaft;
f) locking clamp that allows for the distance between the cuffs to be shortened after the application of the second cuff and to prevent loosening of the flexible cable;
g) a key to release the cuffs and loosen the flexible cable.

2. The invention of claim 1, whereby said hollow shaft is collapsible or extendible.

3. The invention of claim 1, whereby said detachable locking cuffs have an embedded GPS device and self contained power source that activates upon deployment of the cuffs and communicates location of the detainee through a cellular network to a law enforcement server or mobile app.

4. The invention of claim 1, whereby said handle trigger shoots the detachable locking cuffs as projectiles from the end of the hollow shaft.

5. The invention of claim 1, whereby the said detachable locking cuffs are made from a flexible material with means of locking upon deployment.

6. The invention of claim 1, whereby the deployment of the first cuff releases the collapsible second detachable locking cuff to the deployable position by means of spring action.

7. The invention of claim 1, whereby more than two cuffs can be deployed in succession.

8. The invention of claim 1, whereby the cuff trigger contains a sedative to further immobilize the detainee.
